(19) United States
(12) Patent Application Publication
(10) Pub. No.: US 2026/0008983 A1
(43) Pub. Date: Jan. 8, 2026

(54) CULTURE STRUCTURE, CULTURE METHOD, AND CULTURE CHIP

(71) Applicant: BOE TECHNOLOGY GROUP CO., LTD., Beijing (CN)

(72) Inventors: Shicai Wang, Beijing (CN); Yaqi Zhang, Beijing (CN)

(73) Assignee: BOE TECHNOLOGY GROUP CO., LTD., Beijing (CN)

(21) Appl. No.: 18/993,647

(22) PCT Filed: May 17, 2024
(86) PCT No.: PCT/CN2024/093829

§ 371 Date: Jan. 13, 2025

(30) Foreign Application Priority Data
Jun. 30, 2023 (CN) 202310806487.1

(51) Int. Cl. C12M 1/32; C12M 3/06

(52) U.S. Cl. CPC C12M 23/12; C12M 27/16

(57) ABSTRACT

A culture structure, a culture method and a culture chip are provided by embodiments of the present application, the culture structure includes: a culture plate and a vibration signal generating component. By using the culture structure, the culture solution can move and the oxygen and nutrients in the culture solution are balanced. In addition, by using the culture structure, the step of transferring a to-be-cultured object at different growth stages can be avoided and the damage of the transfer to the to-be-cultured object is avoided.

### CROSS-REFERENCE TO RELATED APPLICATION

This application claims the priority of the Chinese Patent application filed on Jun. 30, 2023 before the China National Intellectual Property Administration with the application number of 202310806487.1, and the title of “CULTURE STRUCTURE, CULTURE METHOD, AND CULTURE CHIP”, which is incorporated herein in its entirety by reference.

### FIELD

The present application relates to, but is not limited to, the field of biotechnology and, more particularly, to a culture structure, a culture method, and a culture chip.

### BACKGROUND

In recent years, with the development of cell biology and tissue engineering, three-dimensional cell models are gradually replacing the traditional two-dimensional cell models. Organoids, as a new three-dimensional in vitro research model, are self-assembled by stem cells in vitro, and grow and develop into three-dimensional aggregates similar to the structure and function of human tissues or organs, such as brain organoids, vascular organoids, liver organoids, kidney organoids, and tumor organoids.

### SUMMARY

In order to achieve the above-mentioned object, the following technical solutions are adopted by the embodiments of the present application.

In a first aspect, a culture structure is provided by embodiments of the present application, wherein the culture structure includes: a culture plate and a vibration signal generating component, wherein the vibration signal generating component is disposed on the culture plate;
the culture plate includes a plurality of accommodating structures; the plurality of accommodating structures are configured to accommodate culture solution, and the plurality of accommodating structures include a first accommodating cavity and at least one second accommodating cavity located at a bottom of the first accommodating cavity; a volume of the first accommodating cavity is greater than a volume of the at least one second accommodating cavity; and
the vibration signal generating component is configured to generate a vibration signal; an orthographic projection of the vibration signal generating component on the culture plate does not overlap with the plurality of accommodating structures, and the vibration signal is for driving the culture solution in the plurality of accommodating structures to move.

In at least one embodiment of the present application, the culture plate includes a main body structure and an extending portion located at at least one side of the main body structure; the plurality of accommodating structures are disposed on the main body structure, and the vibration signal generating component is disposed on the extending portion; and
the extending portion is connected to the main body structure, and the vibration signal acts on the main body structure through the extending portion.

In at least one embodiment of the present application, the vibration signal generating component includes at least two piezoelectric transducers; the at least two piezoelectric transducers are disposed on the extending portion, and propagation directions of vibration signals generated by the at least two piezoelectric transducers intersect in a plane where the culture plate is located.

In at least one embodiment of the present application, the vibration signal generating component includes two piezoelectric transducers, and in the plane where the culture plate is located, an included angle formed by the propagation directions of the vibration signals generated by the two piezoelectric transducers is greater than or equal to 80° and less than or equal to 100°.

In at least one embodiment of the present application, the culture plate includes two extending portions, and the two extending portions are located at two adjacent sides of the main body structure, and the two extending portions are connected.

In at least one embodiment of the present application, the culture plate includes a first surface, the first accommodating cavity include a bottom and an opening, and the second accommodating cavity include a bottom and an opening; a plane where the opening of the first accommodating cavity is located is coplanar with the first surface, and a plane where the opening of the second accommodating cavity is located is coplanar with the bottom of the first accommodating cavity;
wherein in a direction perpendicular to the first surface, a distance from the opening of the first accommodating cavity to the bottom of the first accommodating cavity is greater than a distance from the opening of the second accommodating cavity to the bottom of the second accommodating cavity.

In at least one embodiment of the present application, the second accommodating cavity includes a second axis, and an included angle between the second axis of the second accommodating cavity and the first surface is greater than 0° and less than or equal to 90°.

In at least one embodiment of the present application, the included angle between the second axis of the second accommodating cavity and the first surface is substantially 90°, and an area of an orthographic projection of the bottom of the second accommodating cavity on the first surface is less than an area of a region enclosed by an orthographic projection of an edge of the opening of the second accommodating cavity on the first surface.

In at least one embodiment of the present application, the included angle between the second axis of the second accommodating cavity and the first surface is greater than 0° and less than 90°, and an area of an orthographic projection of the bottom of the second accommodating cavity on the first surface is less than or equal to an area of a region enclosed by an orthographic projection of an edge of the opening of the second accommodating cavity on the first surface.

In at least one embodiment of the present application, the first accommodating cavity includes a first axis, and an included angle between the first axis of the first accommodating cavity and the first surface is substantially equal to the included angle between the second axis of the second accommodating cavity and the first surface; an area of a region enclosed by an orthographic projection of an outer contour of the bottom of the first accommodating cavity on the first surface is less than or equal to an area of a region enclosed by an orthographic projection of an edge of the opening of the first accommodating cavity on the first surface.

In at least one embodiment of the present application, an edge of the opening of the first accommodating cavity and an edge of the opening of the second accommodating cavity are arcs, an outer contour of the bottom of the first accommodating cavity and an outer contour of the bottom of the second accommodating cavity are arcs, and a side wall of the first accommodating cavity and a side wall of the second accommodating cavity are arc surfaces.

In at least one embodiment of the present application, the edge of the opening of the first accommodating cavity and the edge of the opening of the second accommodating cavity are circles, and the outer contour of the bottom of the first accommodating cavity and the outer contour of the bottom of the second accommodating cavity are circles;
each of the first accommodating cavity and the second accommodating cavity includes two first regions; an intensity of the vibration signal received by the culture solution in each of the two first regions of the first accommodating cavity is greater than an intensity of the vibration signal received by the culture solution in other regions of the first accommodating cavity; an intensity of the vibration signal received by the culture solution in each of the two first regions of the second accommodating cavity is greater than an intensity of the vibration signal received by the culture solution in other regions of the second accommodating cavity; each circle includes a first diameter and a second diameter, and the first diameter and the second diameter are orthogonal; an extending direction of the first diameter is consistent with the propagation direction of the vibration signal generated by one of the two piezoelectric transducers, and an extending direction of the second diameter is consistent with the propagation direction of the vibration signal generated by the other piezoelectric transducer; and
the two first regions are located at a same side of the first diameter, and orthographic projections of the two first regions on the first surface are symmetrically distributed about the second diameter.

In at least one embodiment of the present application, in the direction perpendicular to the first surface, the distance from the opening of the first accommodating cavity to the bottom of the first accommodating cavity ranges from 250 μm to 1,000 μm, and the distance from the opening of the second accommodating cavity to the bottom of the second accommodating cavity ranges from 200 μm to 300 μm; and
a diameter of a pattern enclosed by the outer contour of the bottom of the first accommodating cavity ranges from 3 mm to 5 mm, and a diameter of a pattern enclosed by the outer contour of the bottom of the second accommodating cavity ranges from 600 μm to 1,000 μm.

In at least one embodiment of the present application, an input voltage signal of the piezoelectric transducer is 0.3 V-0.8 V, and a frequency of the input voltage signal is 150 kHz-400 kHz and 700 kHz-1,200 kHz.

In at least one embodiment of the present application, the culture structure further includes an enclosure structure; a part of the enclosure structure is located between the main body structure and the extending portion, and the enclosure structure is disposed around the plurality of accommodating structures.

In at least one embodiment of the present application, a thickness of the culture plate is 1.8 mm-3.5 mm, a size of the enclosure structure in a thickness direction of the culture plate is 7 mm-12 mm, and a size of the enclosure structure in a direction parallel to the plane where the culture plate is located is 1.5 mm-3.5 mm.

In a second aspect, a culture method is provided by the embodiments of the present application, which is applied to the culture structure according to any one of embodiments in the first aspect, wherein the culture structure includes the culture plate and the vibration signal generating component, and the vibration signal generating component is disposed on the culture plate; the culture plate includes the plurality of accommodating structures; the plurality of accommodating structures are configured to accommodate the culture solution and the plurality of accommodating structures include the first accommodating cavity and the at least one second accommodating cavity located at the bottom of the first accommodating cavity; the orthographic projection of the vibration signal generating component on the culture plate does not overlap with the plurality of accommodating structures; the method includes:
generating the vibration signal; and
driving, by the vibration signal, the culture solution in the plurality of accommodating structures to move.

In at least one embodiment of the present application, the step of driving, by the vibration signal, the culture solution in the plurality of accommodating structures to move includes:
in a first stage, configuring the second accommodating cavity to further accommodate a to-be-cultured object, a frequency of an input voltage signal of the vibration signal generating component being 150 kHz-400 kHz, and driving, by the vibration signal, the to-be-cultured object and the culture solution to move; and
in a second stage, configuring the first accommodating cavity to accommodate the to-be-cultured object, the frequency of the input voltage signal of the vibration signal generating component being 700 kHz-1,200 kHz, and driving, by the vibration signal, the to-be-cultured object and the culture solution to move.

In a third aspect, a culture chip is provided by the embodiments of the present application, wherein the culture chip includes the culture structure according to any one of embodiments in the first aspect.

The above description is only an overview of the technical solution of the present disclosure, in order to be able to better understand the technical means of the present disclosure, and the solution can be implemented in accordance with the content of the description, and in order to make the above and other purposes, features and advantages of the present disclosure more obvious and easy to understand, the following specific embodiments of the present disclosure are hereby given.

### BRIEF DESCRIPTION OF THE DRAWINGS

In order to more clearly illustrate the technical solutions of the embodiments of the present application or the related art, the drawings that are required to be used in the description of the embodiments of the present application or the related art will be briefly introduced below. Apparently, the drawings that are described below are only some embodiments of the present application, and a person skilled in the art can obtain other drawings according to these drawings without paying creative work.

FIGS. 1 and 2 are schematic perspective views of two culture structures according to embodiments of the present application;

FIGS. 3 to 9 are schematic diagrams of seven cross-sectional structures along an M1M2 direction in FIG. 2;

FIG. 10 is a schematic diagram of a cross-sectional structure along an M3M4 direction in FIG. 2;

FIG. 11 is a schematic diagram of a simulation result of an acoustic streaming field of a culture structure according to an embodiment of the present application;

FIG. 12 is a schematic diagram of another simulation result of an acoustic streaming field of a culture structure according to an embodiment of the present application;

FIG. 13 is a schematic diagram of a simulation result of a particle tracking trajectory in an acoustic streaming field of a culture structure according to an embodiment of the present application; and

FIGS. 14A, 14B, and 14C are schematic top views of three accommodating structures according to embodiments of the present application.

### DETAILED DESCRIPTION

The technical solutions of the embodiments of the present application will be clearly and completely described below with reference to the drawings of the embodiments of the present application. Apparently, the described embodiments are merely certain embodiments of the present application, rather than all of the embodiments. All of the other embodiments that a person skilled in the art obtains on the basis of the embodiments of the present application without paying creative work fall within the protection scope of the present application.

In the embodiments of the present application, terms such as “first”, “second”, “third” and “fourth” are used to distinguish identical items or similar items that have substantially the same functions and effects, merely in order to clearly describe the technical solutions of the embodiments of the present application, and should not be construed as indicating or implying the degrees of importance or implicitly indicating the quantity of the specified technical features.

In the embodiments of the present application, the terms that indicate orientation or position relations, such as “upper” and “lower”, are based on the orientation or position relations shown in the drawings, and are merely for conveniently describing the present application and simplifying the description, rather than indicating or implying that the device or element must have the specific orientation and be constructed and operated according to the specific orientation. Therefore, they should not be construed as a limitation on the present application.

In the description of the present disclosure, the terms “one embodiment”, “some embodiments”, “exemplary embodiment”, “example”, “specific example” or “some examples” are intended to indicate that specific features, structures, materials or characteristics related to the embodiment or example are included in at least one embodiment or example of the present application. The illustrative indication of the above terms does not necessarily refer to the same embodiment or example. Moreover, the specific features, structures, materials or characteristics may be included in any one or more embodiments or examples in any suitable manner.

In the embodiments of the present application, the meaning of “plurality of” is “two or more”, and the meaning of “at least one” is “one or more”, unless explicitly and particularly defined otherwise.

All of the features used in the embodiments of the present application of “parallel”, “perpendicular”, “the same” and so on include the features of “parallel”, “perpendicular”, “the same” and so on in the strict sense, and include the cases in which there is a certain tolerance such as “substantially parallel”, “substantially perpendicular” and “substantially the same”, taking into consideration the measurement and the tolerances relevant to the measurement on particular quantities (for example, restricted by the measuring system), and represent that they are in the acceptable deviation ranges of the particular values determined by a person skilled in the art. For example, the “substantially” can represent that they are within one or more standard deviations, or within 10% or 5% of the values.

Unless stated otherwise in the context, throughout the description and the claims, the term “include” is interpreted as the meaning of opened accommodating, i.e., “including but not limited to”. The polygons used herein are not polygons in strict sense, may be an approximate triangle, parallelogram, trapezoid, pentagon, hexagon and so on, and may have some tiny deformations caused by tolerances.

With the development of the understanding of diseases, the research on complex diseases such as major nervous system diseases has progressed from the single-cell level to the multi-cellular and even tissue and organ levels. Based on the induced pluripotent stem cells (iPSCs), simulating the development of organoids in vitro for disease and drug research has gradually become a hot spot in current research and industry.

However, during the culture process of organoids, uneven growth and development of organoids often occur, and organoids are prone to lack of oxygen or nutrients in the center during growth resulting in incomplete development or even death of organoid cells. Therefore, it is necessary to introduce a physical method to change the distribution of environmental nutrients, thereby the growth of the organoids cells is ensured. Organoid refers to the organ-like tissue structure with relatively stable phenotype and genetic characteristics cultured in vitro by using the 3D culture technology, which is of great significance in the study of growth and development, physiology and pathology, drug effects, and so on.

It should be noted that the resources of genetic characteristics involved in the present specification are genetic resources that are properly acquired or utilized in accordance with the laws and administrative law regulations.

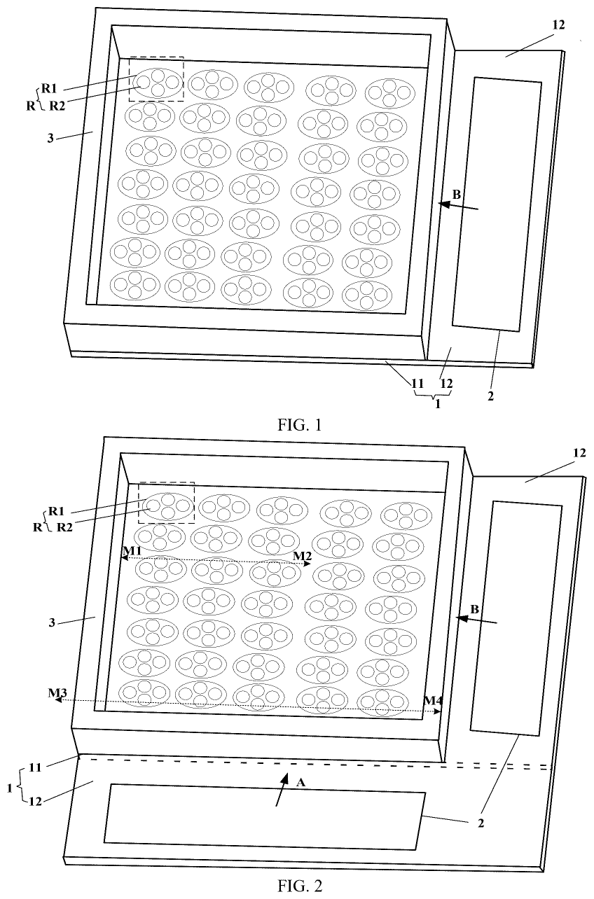

Therefore, a culture structure is provided by embodiments of the present application, as shown in FIG. 1 or FIG. 2, the culture structure includes: a culture plate 1 and a vibration signal generating component (such as a piezoelectric transducer 2). The vibration signal generating component is disposed on the culture plate 1.

The culture plate 1 includes a plurality of accommodating structures R. The plurality of accommodating structures R are configured to accommodate culture solution and include a first accommodating cavity R1 and at least one second accommodating cavity R2 located at a bottom of the first accommodating cavity R1. A volume of the first accommodating cavity R1 is greater than a volume of the at least one second accommodating cavity R2.

The vibration signal generating component 2 is configured to generate a vibration signal. An orthographic projection of the vibration signal generating component on the culture plate 1 does not overlap with the plurality of accommodating structures R. The vibration signal is for driving the culture solution in the plurality of accommodating structures R to move.

In embodiments of the present application, the culture structure may be configured to culture organoids or cells.

According to the culture structure provided by the embodiments of the present application, the vibration signal generated by the vibration signal generating component drives the culture solution in the plurality of accommodating structures R to move. During the organoid growth process, sufficient oxygen may be provided to the organoids, and the oxygen and nutrients in the culture solution are balanced so that the organoids cultured in the culture solution develop completely, thereby the technical problem of organoid cell death caused by lack of oxygen or nutrients in the center during the organoid growth process is solved.

In an exemplary embodiment, the vibration signal generated by the vibration signal generating component may include sound waves, such as ultrasonic waves. Specifically, the transmission of sound waves into the culture solution may generate an acoustic streaming field, the acoustic streaming field may drive the culture solution in the accommodating structure R to move.

When the organoid is placed in the culture solution, the organoid is driven to rotate by the culture solution. A main acting force on the organoid is a drag force, and the calculation formula is as follows:

$${F}_{drag} = - 6 πμ a ( \vec{{v}_{p}} - \vec{{v}_{f}} )$$

wherein μ is a dynamic viscosity coefficient of the fluid, a is a cell radius (organoid radius), \vec{vₚ} is a cell velocity (organoid velocity), and \vec{v_{f}} is a liquid velocity. The drag force is the force exerted by a fluid on a solid with a relative velocity. This force is in the opposite direction to the velocity of the solid relative to the fluid and is essentially the resistance to the relative motion. In an exemplary embodiment, since the second accommodating cavity R2 is disposed at

the bottom of the first accommodating cavity R1, the volume of the second accommodating cavity R2 is less than the volume of the first accommodating cavity R1, and the amount of the culture solution accommodated in the second accommodating cavity R2 is less than the amount of the culture solution accommodated in the first accommodating cavity R1.

In the culture structure provided by the embodiments of the present application, the volume of the second accommodating cavity R2 is less than the volume of the first accommodating cavity R1. Thus, the organoid or tissue with a smaller volume may be cultured in the second accommodating cavity R2, and the organoid or tissue with a larger volume may be cultured in the first accommodating cavity R1. For example, at an initial stage of the organoid development, the organoid is placed in the second accommodating cavity R2, and at a later stage of the organoid development, it is transferred from the second accommodating cavity R2 to the first accommodating cavity R1.

The specific material of the above-mentioned culture plate 1 is not limited herein. Exemplarily, the material of the culture plate 1 may be a transparent or light-transmitting material to facilitate the observation of the organoid growth process.

For example, the above-mentioned transparent or light-transmitting material may include glass, acrylic resin (polymethyl methacrylate (PMMA)), polystyrene (PS), polycarbonate (PC), and polyethylene terephthalate (PET).

In an exemplary embodiment, the vibration signal generating component may be bonded to the culture plate 1.

The arrangement of the plurality of accommodating structures R on the culture plate 1 is not limited herein. Exemplarily, the plurality of accommodating structures R may be arranged in an array.

In the related art, the culture process of the organoid involves transfer through three culture plates. Firstly, small spheres are formed in a 96-well round-bottom culture plate, developed into embryoid bodies, and then transferred to a 24-well plate for further differentiation and development. After further growth, they are transferred to a 6-well plate for the final shaker oscillation development. That is, the traditional culture process of the organoid involves 3 transfers, which is very prone to organoid loss during the transfer processes.

In the embodiment of the present application, since the accommodating structure R includes a first accommodating cavity R1 and at least one second accommodating cavity R2 located at the bottom of the first accommodating cavity R1, it is possible to carry out the multi-stage culture of the organoid in the culture structure during the organoid growth process, thereby the loss and damage of the organoid caused by the transfer of the organoid at different development stages are avoided and the culture efficiency is improved.

The accommodating structure R (including the first accommodating cavity R1 and the second accommodating cavity R2) in the culture structure provided by the embodiment of the present application may be adjusted to its corresponding size and shape according to actual requirements, thereby various types of application scenarios are matched, for example, the culture of different types of organoids is matched. The culture structure of the embodiment of the present application has a simple overall structure and a low preparation cost, and may be prepared in batches by using a processing process, which facilitates large-scale popularization and application.

In at least one embodiment of the present application, as shown in FIGS. 1 and 2, the culture plate 1 includes a main body structure 11 and an extending portion 12 located at at least one side of the main body structure 11. The plurality of accommodating structures R are disposed on the main body structure 11, and the vibration signal generating component is disposed on the extending portion 12. The extending portion 12 is connected to the main body structure 11, and the vibration signal acts on the main body structure 11 through the extending portion 12.

A planar shape of the main body structure 11 in the above-mentioned culture plate 1 and a planar shape of the extending portion 12 in the above-mentioned culture plate 1 are not limited herein. Exemplarily, the planar shape of the main body structure 11 and the planar shape of the extending portion 12 may be arcs, polygons, or a combination of arcs and polygons.

For example, the planar pattern of the main body structure 11 and the planar pattern of the extending portion 12 may be polygons, including, but not limited to, quadrangles, pentagons, and the like. Polygons in the present specification may include standard polygons and non-standard polygons, wherein the non-standard polygons may include rounded polygons.

For example, the planar pattern of the main body structure 11 and the planar pattern of the extending portion 12 may be arcs, including, but not limited to, circles and ovals.

In an exemplary embodiment, the extending portion 12 and the main body structure 11 may be made of the same material in the same preparation process, and the two may be integrated as a whole.

In the embodiment of the present application, the extending portion 12 is connected to the main body structure 11, and the vibration signal acts on the main body structure 11 through the extending portion 12. Since the plurality of accommodating structures R are disposed on the main body structure 11, the vibration signal may be propagated to the culture solution through the main body structure 11, thereby the culture solution in the plurality of accommodating structures R is driven to move, the oxygen in the culture solution is improved, and the oxygen and nutrients in the culture solution are enabled to be evenly distributed.

When the extending portion 12 and the main body structure 11 are integrated as a whole, the propagation of the vibration signal is further facilitated, and the loss of the vibration signal during propagation is reduced.

In at least one embodiment of the present application, as shown in FIG. 2, the vibration signal generating component includes at least two piezoelectric transducers 2. The at least two piezoelectric transducers 2 are disposed on the extending portions 12, and propagation directions of vibration signals generated by the at least two piezoelectric transducers 2 intersect in a plane where the culture plate 11 is located.

A core component of the piezoelectric transducer includes a piezoelectric crystal plate. The piezoelectric crystal plate may be deformed under pressure, leading to the polarization of the crystal plate itself and the appearance of positive and negative bound charges on the surface of the crystal plate. This effect is referred to as a piezoelectric effect. In addition, the piezoelectric effect is reversible, i.e., when a voltage is applied to the crystal plate, deformation occurs, thereby a vibration signal is generated. The vibration modes may include a stretching vibration, a bending vibration and a torsional vibration.

The planar shape of the piezoelectric transducer includes, but is not limited to, a rectangle, a circle, a circular ring and a circular tube.

In at least one embodiment of the present application, as shown in FIG. 2, the vibration signal generating component includes two piezoelectric transducers 2, and in the plane where the culture plate 1 is located, an included angle (such as an included angle between direction A and direction B) formed by the propagation directions of the vibration signals generated by the two piezoelectric transducers 2 is greater than or equal to 80° and less than or equal to 100°.

Exemplarily, the included angle formed by the propagation directions of the vibration signals generated by the two piezoelectric transducers 2 may be 80°, 85°, 88°, 90°, 93°, 95°, 98°, and 100°.

For example, the included angle formed by the propagation directions of the vibration signals generated by the two piezoelectric transducers 2 is 90°. i.e., the propagation directions of the vibration signals generated by the two piezoelectric transducers 2 are orthogonal in the plane where the culture plate 1 is located.

In the embodiment of the present application, the included angle (such as the included angle between direction A and direction B) formed by the propagation directions of the vibration signals generated by the two piezoelectric transducers 2 is greater than or equal to 80° and less than or equal to 100°. Thus, for the culture solution in the same accommodating structure R, the vibration signals received are asymmetrical, which causes the culture solution in the accommodating structure R to move from a region where the received vibration signal is strong to a region where the received vibration signal is weak, thereby the culture solution is driven to move.

It should be noted that when the propagation directions of the vibration signals generated by the two piezoelectric transducers 2 are disposed to be parallel and opposite, the vibration signals generated by the two piezoelectric transducers 2 are canceled in the culture solution, and the culture solution may fluctuate but is difficult to move so that it is difficult to achieve uniform distribution of nutrients and oxygen in the culture solution.

In the embodiment of the present application, the above-mentioned two piezoelectric transducers 2 have the same parameter settings when generating vibration signals. For example, the two piezoelectric transducers 2 have the same model and may use the same input voltage, the same frequency and the same phase. When it is necessary to adjust the frequency of the piezoelectric transducer 2 to change the size of the vibration signal during the actual culture of the organoid, the two piezoelectric transducers 2 are adjusted simultaneously.

In the related art, by using four interdigital transducers disposed at the periphery of the accommodating structure of the culture plate, and the frequency and phase of each interdigital transducer are adjusted, so that the culture solution in the accommodating structure undergoes a double rotation movement in a plane formed by a Y direction and a Z direction (a plane of a vertical direction). However, vibration signals generated by two interdigital transducers disposed opposite to each other are easily canceled, and the process of adjusting the frequencies and phases of the four interdigital transducers is relatively complicated, which increases the difficulty of the experimental operation in the culture process of the organoid.

In the embodiment of the present application, by setting that the two piezoelectric transducers 2 have the same parameter settings when generating the vibration signals, the step of culturing the organoid using the culture structure can be greatly simplified, and the difficulty of the culture process can be reduced. In addition, the culture structure of the present application can generate a reflux field in a plane formed by an X direction and a Y direction (a plane of a horizontal direction) and drive the culture solution and the organoid to move (referring to the description below for details). Meanwhile, the quantity of the transducers used is small, thereby the cost is reduced.

In at least one embodiment of the present application, as shown in FIG. 2, the culture plate 1 includes two extending portions 12. The two extending portions 12 are located at two adjacent sides of the main body structure 11, and the two extending portions 12 are connected.

In an exemplary embodiment, the planar pattern of the main body structure 11 is a rectangle. At this moment, the two extending portions 12 are located at two adjacent sides of the main body structure 11, and one piezoelectric transducer 2 is disposed on one extending portion 12, so that it is possible to realize that the included angle (such as the included angle between direction A and direction B) formed by the propagation directions of the vibration signals generated by the two piezoelectric transducers 2 is greater than or equal to 80° and less than or equal to 100°.

In the embodiment of the present application, by disposing that the two extending portions 12 located at two adjacent sides of the main body structure 11 are connected together, the mechanical stability of the extending portions 12 and the main body structure 11 can be improved, thereby damage to the culture plate 1 caused by the high-frequency operation of the piezoelectric transducers 2 is avoided, and the service life of the culture structure is improved.

In the embodiment of the present application, the extending portion 12 serves as a transmission medium of the vibration signal and transmits the vibration signal to the main body structure 11 and then to the culture solution in the accommodating structure R. In addition, the propagation directions of the vibration signals generated by the two piezoelectric transducers 2 intersect in the plane where the culture plate 11 is located, thereby the construction of the reflux field of the culture solution in the accommodating structure R (including the first accommodating cavity R1 and the second accommodating cavity R2) is realized. Thus, particles in the culture solution flow back from the region where the vibration signal is strong to the region where the vibration signal is weak, so that the uniform rotation of the culture solution is ensured. The constant rotation of the fluid ensures the uniform distribution of nutrients and oxygen, thereby sufficient supply is provided to the organoid and the survival rate of the organoid is improved to solve problems such as hypoxia of the organoid in the culture solution on the culture plate.

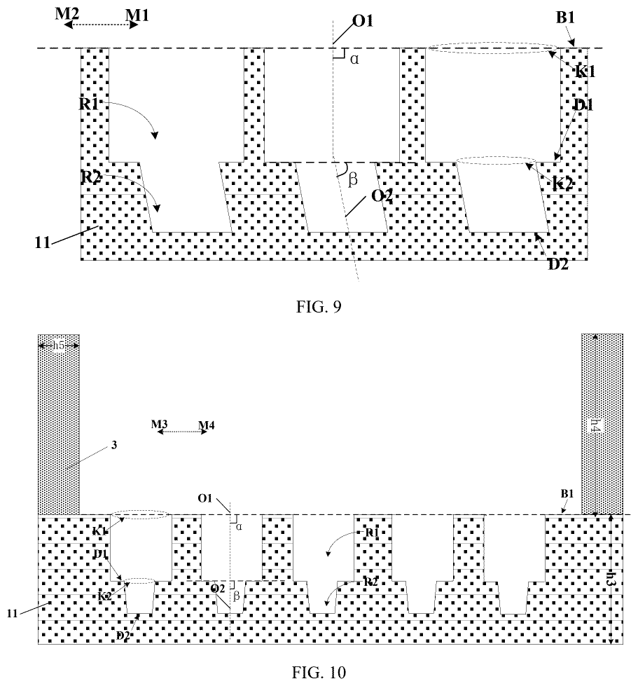

In at least one embodiment of the present application, as shown in FIGS. 3 to 10, the culture plate 1 includes a first surface B1, the first accommodating cavity R1 includes a bottom and an opening, and the second accommodating cavity R2 includes a bottom and an opening. A plane where the opening K1 of the first accommodating cavity R1 is located is coplanar with the first surface B1, and a plane where the opening K2 of the second accommodating cavity R2 is located is coplanar with the bottom D1 of the first accommodating cavity R1.

As shown in FIG. 3, in a direction perpendicular to the first surface B1, a distance h1 (a depth of the first accommodating cavity R1) from the opening K1 of the first accommodating cavity R1 and the bottom D1 of the first accommodating cavity R1 is greater than a distance h2 (a depth of the second accommodating cavity R2) from the opening K2 of the second accommodating cavity R2 to the bottom D2 of the second accommodating cavity R2.

In the embodiment of the present application, at the initial stage of the organoid development, the organoid is placed in the second accommodating cavity R2, and at the later stage of the organoid development, the organoid is transferred from the second accommodating cavity R2 to the first accommodating cavity R1. By setting that the depth of the first accommodating cavity R1 is greater than the depth of the second accommodating cavity R2, i.e., the depth of the second accommodating cavity R2 is less than the depth of the first accommodating cavity R1, during the actual culture of the organoid, it is more conducive to transfer the organoid from the second accommodating cavity R2 to the first accommodating cavity R1 under the action of the buoyancy and the reflux field (acoustic streaming field) generated by the vibration signal to avoid the death problem caused by the oversize organoid and hypoxia due to transfer difficulty.

In at least one embodiment of the present application, as shown in FIGS. 3 to 9, the second accommodating cavity R2 includes a second axis O2, and an included angle β between the second axis O2 of the second accommodating cavity R2 and the first surface B1 is greater than 0° and less than or equal to 90°.

It should be noted that, in the present specification, the included angle β between the second axis O2 of the second accommodating cavity R2 and the first surface B1 refers to a smaller included angle between the second axis O2 of the second accommodating cavity R2 and the first surface B1.

In an exemplary embodiment, the first surface B1 and a surface of the culture solution are substantially parallel.

In the culture structure provided by the embodiment of the present application, the first accommodating cavity R1 and the second accommodating cavity R2 may each have an axisymmetric structure. The second axis O2 is a symmetry axis of the second accommodating cavity R2.

In some embodiments, as shown in FIGS. 3, 4 and 5, the included angle β between the second axis O2 of the second accommodating cavity R2 and the first surface B1 is substantially equal to 90°, i.e., an extending direction of the second accommodating cavity R2 is substantially perpendicular to the surface of the culture solution.

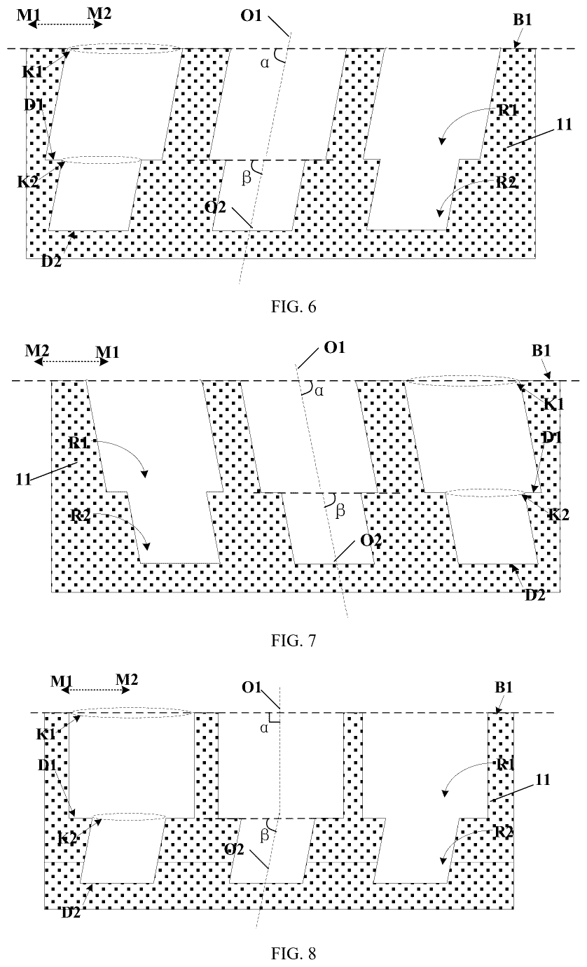

In some embodiments, as shown in FIGS. 6, 7, 8 and 9, the included angle β between the second axis O2 of the second accommodating cavity R2 and the first surface B1 is less than 90°. That is, there is an acute angle between the extending direction of the second accommodating cavity R2 and the surface of the culture solution, and the second accommodating cavity R2 is inclined with respect to the surface of the culture solution.

In the culture structure provided by the embodiment of the present application, the second accommodating cavity R2 is inclined with respect to the surface of the culture solution. Thus, when the organoid is transferred from the second accommodating cavity R2 to the first accommodating cavity R1, it is more conducive to transfer the organoid from the second accommodating cavity R2 to the first accommodating cavity R1 under the action of the buoyancy and the reflux field (acoustic streaming field) generated by the vibration signal to avoid the death problem caused by the oversize organoid and hypoxia due to transfer difficulty.

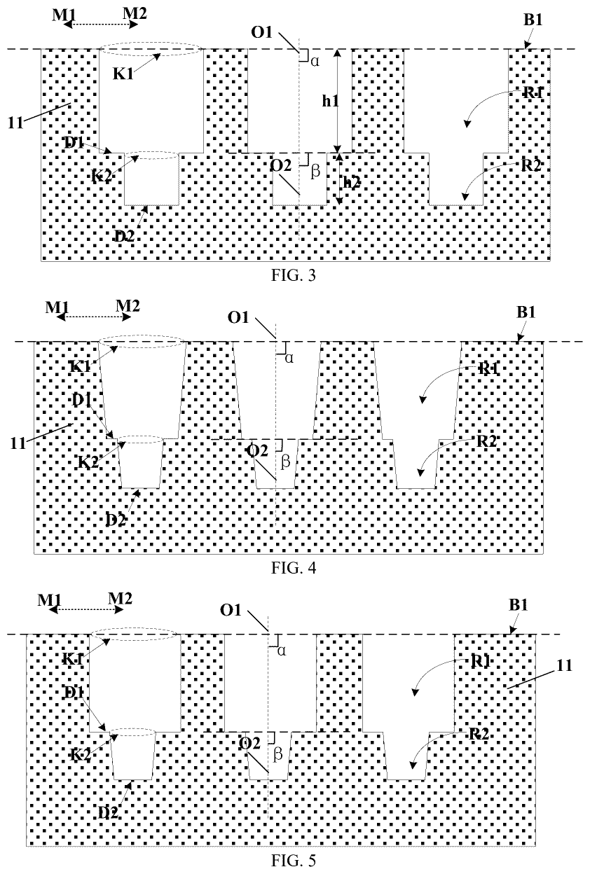

In at least one embodiment of the present application, as shown in FIGS. 4 and 5, the included angle between the second axis O2 of the second accommodating cavity R2 and the first surface B1 is substantially 90°, and an area of an orthographic projection of the bottom D2 of the second accommodating cavity R2 on the first surface B1 is less than an area of a region enclosed by an orthographic projection of an edge of the opening K2 of the second accommodating cavity R2 on the first surface B1.

In an exemplary embodiment, the above-mentioned edge of the opening K2 may also be referred to as a border of the opening K2. Similar descriptions hereinafter have the same meaning as here, and will not be described again.

In the culture structure provided by the embodiment of the present application, by setting that the area of the orthographic projection of the bottom D2 of the second accommodating cavity R2 on the first surface B1 is less than the area of the region enclosed by the orthographic projection of the edge of the opening K2 of the second accommodating cavity R2 on the first surface B1, the second accommodating cavity R2 has a structure with a small bottom D2 and a large opening K2. When the organoid is transferred from the second accommodating cavity R2 to the first accommodating cavity R1, it is more conducive to transfer the organoid from the second accommodating cavity R2 to the first accommodating cavity R1 under the action of the buoyancy and the reflux field (acoustic streaming field) generated by the vibration signal to avoid the death problem caused by the oversize organoid and hypoxia due to transfer difficulty.

In at least one embodiment of the present application, as shown in FIGS. 6, 7, 8 and 9, the included angle between the second axis of the second accommodating cavity and the first surface is greater than 0° and less than 90°, and an area of an orthographic projection of the bottom D2 of the second accommodating cavity R2 on the first surface B1 is less than or equal to the area of the region enclosed by the orthographic projection of an edge of the opening K2 of the second accommodating cavity R2 on the first surface B1.

Exemplarily, when the included angle between the second axis of the second accommodating cavity and the first surface is greater than 0° and less than 90° (i.e., the second accommodating cavity R2 is inclined with respect to the surface of the culture solution), the area of the orthographic projection of the bottom D2 of the second accommodating cavity R2 on the first surface B1 may be set to be less than the area of the region enclosed by the orthographic projection of the edge of the opening K2 of the second accommodating cavity R2 on the first surface B1, or the area of the orthographic projection of the bottom D2 of the second accommodating cavity R2 on the first surface B1 may be set to be equal to the area of the region enclosed by the orthographic projection of the edge of the opening K2 of the second accommodating cavity R2 on the first surface B1.

When it is ensured that the organoid can be smoothly transferred from the first accommodating cavity R1 to the second accommodating cavity R2, in order to simplify the design of the culture structure and reduce the difficulty of the preparation process thereof, when the second accommodating cavity R2 is inclined with respect to the surface of the culture solution, the area of the orthographic projection of the bottom D2 of the second accommodating cavity R2 on the first surface B1 may be set to be equal to the area of the region enclosed by the orthographic projection of the edge of the opening K2 of the second accommodating cavity R2 on the first surface B1.

In the drawings provided by the embodiments of the present application, the figures are drawn by taking that the area of the orthographic projection of the bottom D2 of the second accommodating cavity R2 on the first surface B1 is equal to the area of the region enclosed by the orthographic projection of the edge of the opening K2 of the second accommodating cavity R2 on the first surface B1 as an example.

In at least one embodiment of the present application, as shown in FIGS. 3, 4, 5, 6 and 7, the first accommodating cavity R1 includes a first axis O1, and an included angle α between the first axis O1 of the first accommodating cavity R1 and the first surface B1 is substantially equal to the included angle β between the second axis O2 of the second accommodating cavity R2 and the first surface B1. An area of a region enclosed by an orthographic projection of an outer contour of the bottom D1 of the first accommodating cavity R1 on the first surface B1 is less than or equal to an area of a region enclosed by an orthographic projection of an edge of the opening K1 of the first accommodating cavity R1 on the first surface B1.

In an exemplary embodiment, as shown in FIGS. 3 and 5, the first accommodating cavity R1 includes a first axis O1, and the included angle α between the first axis O1 of the first accommodating cavity R1 and the first surface B1 and the included angle β between the second axis O2 of the second accommodating cavity R2 and the first surface B1 are substantially 90°. The area of the region enclosed by the orthographic projection of the outer contour of the bottom D1 of the first accommodating cavity R1 on the first surface B1 is equal to the area of the region enclosed by the orthographic projection of the edge of the opening K1 of the first accommodating cavity R1 on the first surface B1.

In an exemplary embodiment, as shown in FIG. 4, the first accommodating cavity R1 includes a first axis O1, and the included angle α between the first axis O1 of the first accommodating cavity R1 and the first surface B1 and the included angle β between the second axis O2 of the second accommodating cavity R2 and the first surface B1 are substantially 90°. The area of the region enclosed by the orthographic projection of the outer contour of the bottom D1 of the first accommodating cavity R1 on the first surface B1 is less than the area of the region enclosed by the orthographic projection of the edge of the opening K1 of the first accommodating cavity R1 on the first surface B1.

In an exemplary embodiment, as shown in FIGS. 6 and 7, the first accommodating cavity R1 includes a first axis O1, and the included angle α between the first axis O1 of the first accommodating cavity R1 and the first surface B1 is substantially equal to the included angle β between the second axis O2 of the second accommodating cavity R2 and the first surface B1, both of which are less than 90°. The area of the region enclosed by the orthographic projection of the outer contour of the bottom D1 of the first accommodating cavity R1 on the first surface B1 is equal to the area of the region enclosed by the orthographic projection of the edge of the opening K1 of the first accommodating cavity R1 on the first surface B1.

In the culture structure provided by the embodiment of the present application, by setting that the included angle α between the first axis O1 of the first accommodating cavity R1 and the first surface B1 is substantially equal to the included angle β between the second axis O2 of the second accommodating cavity R2 and the first surface B1, i.e., the first axis O1 and the second axis O2 are collinear, when the organoid is transferred from the second accommodating cavity R2 to the first accommodating cavity R1, it is more conducive to transfer the organoid from the second accommodating cavity R2 to the first accommodating cavity R1 under the action of the buoyancy and the reflux field (acoustic streaming field) generated by the vibration signal to avoid the death problem caused by the oversize organoid and hypoxia due to transfer difficulty.

In another embodiment, when the size of the organoid is small or the size of the accommodating structure R is large, the included angle α between the first axis O1 of the first accommodating cavity R1 and the first surface B1 may be different from the included angle β between the second axis O2 of the second accommodating cavity R2 and the first surface B1, i.e., there is an included angle between the first axis O1 and the second axis O2, and the organoid may also be transferred from the second accommodating cavity R2 to the first accommodating cavity R1 under the action of the buoyancy and the acoustic streaming field generated by the vibration signal to avoid the death problem caused by the oversize organoid and hypoxia due to transfer difficulty.

In at least one embodiment of the present application, as shown in FIGS. 14A, 14B and 14C, the edge of the opening K of the first accommodating cavity R1 and the edge of the opening K of the second accommodating cavity R2 are arcs.

In addition, an outer contour of the bottom of the first accommodating cavity R1 and an outer contour of the bottom of the second accommodating cavity R2 are arcs, and a side wall of the first accommodating cavity R1 and a side wall of the second accommodating cavity R2 are arc surfaces.

It should be noted that each of FIGS. 14A, 14B, and 14C shows the contour of the edge of the respective opening K of the culture structure in a top view.

In an exemplary embodiment, a pattern formed by the edge of the opening K of the first accommodating cavity R1 and a pattern formed by the edge of the opening K of the second accommodating cavity R2 are arcs, wherein the arc may include a circle and an oval.

Similarly, a pattern formed by the outer contour of the bottom of the first accommodating cavity R1 and a pattern formed by the outer contour of the bottom of the second accommodating cavity R2 are arcs, for example, circles and ovals.

Whether the pattern formed by the edge of the opening described above is the same as the pattern formed by the outer contour of the bottom is not limited herein and may be specifically determined according to requirements.

In an exemplary embodiment, the first accommodating cavity R1 may have a cylindrical shape, a truncated cone shape, an elliptical cylinder shape, an elliptical truncated cone shape, an oblique cylindrical shape, an oblique truncated cone shape, an oblique elliptical cylindrical shape, or an oblique elliptical truncated cone shape.

In an exemplary embodiment, the second accommodating cavity R2 may have a cylindrical shape, a truncated cone shape, an elliptical cylinder shape, an elliptical truncated cone shape, an oblique cylindrical shape, an oblique truncated cone shape, an oblique elliptical cylindrical shape, or an oblique elliptical truncated cone shape.

In the culture structure provided by the embodiment of the present application, the edge of the opening K of the first accommodating cavity R1 and the edge of the opening K of the second accommodating cavity R2 are arcs, the outer contour of the bottom of the first accommodating cavity R1 and the outer contour of the bottom of the second accommodating cavity R2 are arcs, and the side wall of the first accommodating cavity R1 and the side wall of the second accommodating cavity R2 are arc surfaces. When the moving culture solution drives the organoid to rotate, the loss of the organoid caused by a sharp structure in the first accommodating cavity R1 and the second accommodating cavity R2 is avoided, the loss rate of the organoid is reduced and the survival rate of the organoid is improved while ensuring the uniform absorption of oxygen and nutrients by the organoid.

In at least one embodiment of the present application, the edge of the opening of the first accommodating cavity R1 and the edge of the opening of the second accommodating cavity R2 are circles, and the outer contour of the bottom of the first accommodating cavity R1 and the outer contour of the bottom of the second accommodating cavity R2 are circles.

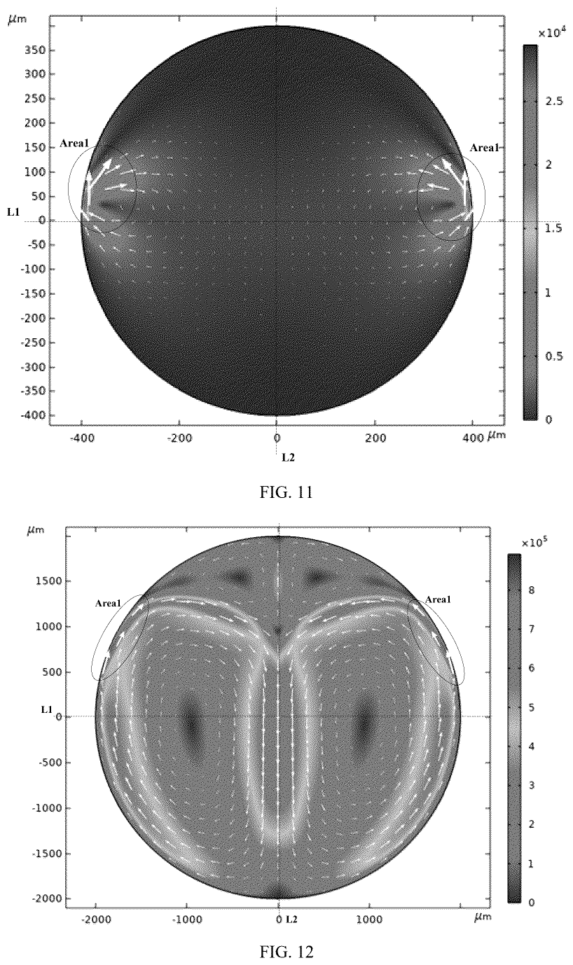

FIG. 11 provides a simulation effect diagram of an acoustic streaming field in two dimensions (the X direction and the Y direction) in the second accommodating cavity R2, and FIG. 12 provides a simulation effect diagram of an acoustic streaming field in two dimensions (the X direction and the Y direction) in the first accommodating cavity R1. The unit of the simulation effect diagram of the acoustic streaming field is mm/s. A region marked with a larger arrow in the drawings indicates a stronger acoustic stream, and the direction of the arrow indicates the direction of the acoustic stream (the flow of the culture solution).

As shown in FIGS. 11 and 12, each of the first accommodating cavity R1 and the second accommodating cavity R2 includes two first regions Area1. Each of the two first regions Area1 of the first accommodating cavity is greater than an intensity of the vibration signal received by the culture solution in other regions of the first accommodating cavity (i.e., the intensity of the acoustic stream and the flow rate of the culture solution in the first region Area1 are greater than the intensity of the acoustic stream and the flow rate of the culture solution in the other regions); an intensity of the vibration signal received by the culture solution in each of the two first regions Area1 of the second accommodating cavity is greater than an intensity of the vibration signal received by the culture solution in other regions of the second accommodating cavity; each circle includes a first diameter L1 and a second diameter L2, and the first diameter L1 and the second diameter L2 are orthogonal; and the two first regions Area1 are located at a same side of the first diameter L1, and orthographic projections of the two first regions Area1 on the first surface B1 are symmetrically distributed about the second diameter L2.

In an exemplary embodiment, as shown in FIG. 11, the intensity of the acoustic streaming field in the two first regions Area1 is greatest, and the culture solution in the second accommodating cavity R2 performs a swirling motion from the outside to the inside and drives the particles or cells in the culture solution to perform a swirling motion from the outside to the inside.

In FIG. 11, the two first regions Area1 are located at an upper side of the first diameter L1. That is, for the culture solution in the second accommodating cavity R2, the culture solution in a left half region rotates clockwise driven by the acoustic streaming field in the first region Area1, and the culture solution in a right half region rotates counterclockwise driven by the acoustic streaming field in the first region Area1. Since the intensity of the acoustic streaming field is distributed asymmetrically in the left half region, the culture solution in the left half region is driven to rotate clockwise. Since the intensity of the acoustic streaming field is distributed asymmetrically in the right half region, the culture solution in the right half region is driven to rotate counterclockwise. That is, the culture solution in the two regions perform a swirling motion from the outside to the inside.

In an exemplary embodiment, as shown in FIG. 12, since the size of the first accommodating cavity R1 is greater than the size of the second accommodating cavity R2, the culture solution accommodated by the first accommodating cavity R1 is more. At this moment, the input frequency in the first accommodating cavity R1 is increased, the tendency of the culture solution in the first accommodating cavity R1 to perform a swirling motion from the outside to the inside is more remarkable. Similarly, in FIG. 12, the two first regions Area1 are located at the upper side of the first diameter L1. That is, for the culture solution in the first accommodating cavity R1, the culture solution in the left half region rotates clockwise driven by the acoustic streaming field in the first region Area1, and the culture solution in the right half region rotates counterclockwise driven by the acoustic streaming field in the first region Area1.

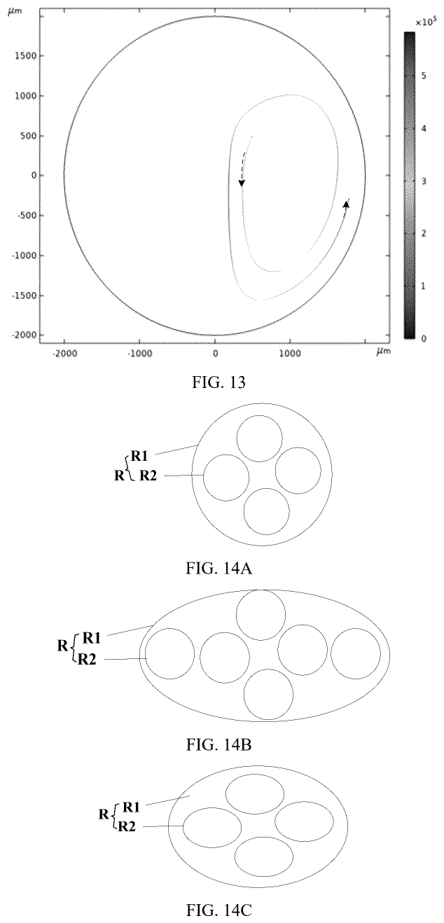

FIG. 13 provides a schematic diagram of a simulation result of a particle tracking trajectory of 500 μm particles in an accommodating structure. The unit is um/s, and the input frequency of the piezoelectric transducer 2 at this moment is 800 kHz. The dotted arrows marked in FIG. 13 indicate a movement direction of the particles. According to the simulation effect of FIG. 13, it can be seen that the particles with a diameter of 500 μm may complete two rotations within 3 s without colliding with the side wall of the accommodating structure. It can be seen that, according to the culture structure provided by the embodiment of the present application, the organoid can rotate well in the culture structure so that during the organoid growth process, the culture solution can uniformly supply oxygen and nutrients to the organoid, thereby the technical problem of organoid cell death caused by lack of oxygen or nutrients in the center during the organoid growth process is solved.

In at least one embodiment of the present application, in the direction perpendicular to the first surface B1, the distance h1 from of the opening K1 the first accommodating cavity R1 to the bottom D1 of the first accommodating cavity R1 ranges from 250 μm to 1,000 μm, and the distance h2 from the opening K2 of the second accommodating cavity R2 to the bottom D2 of the second accommodating cavity R2 ranges from 200 μm to 300 μm.

A diameter of a pattern enclosed by the outer contour of the bottom D1 of the first accommodating cavity R1 ranges from 3 mm to 5 mm, and a diameter of a pattern enclosed by the outer contour of the bottom of the second accommodating cavity R2 ranges from 600 μm to 1,000 μm.

Exemplarily, the diameter of the pattern enclosed by the outer contour of the bottom D1 of the first accommodating cavity R1 may be the same as the diameter of the pattern enclosed by the edge of the opening K1 of the first accommodating cavity R1, or the diameter of the pattern enclosed by the outer contour of the bottom D1 of the first accommodating cavity R1 may be smaller than the diameter of the pattern enclosed by the edge of the opening K1 of the first accommodating cavity R1.

Exemplarily, the diameter of the pattern enclosed by the outer contour of the bottom D2 of the second accommodating cavity R2 may be the same as the diameter of the pattern enclosed by the edge of the opening K2 of the second accommodating cavity R2, or the diameter of the pattern enclosed by the outer contour of the bottom D2 of the second accommodating cavity R2 may be smaller than the diameter of the pattern enclosed by the edge of the opening K2 of the second accommodating cavity R2.

Exemplarily, the distance h1 (i.e., the depth) from the opening K1 of the first accommodating cavity R1 to the bottom D1 of the first accommodating cavity R1 may be 300 μm, 350 μm, 380 μm, 400 μm, 420 μm, 450 μm, 480 μm, 500 μm, 530 μm, 550 μm, 580 μm, 600 μm, 630 μm, 660 μm, 680 μm, 700 μm, 730 μm, 750 μm, 780 μm, 800 μm, 850 μm, 880 μm, 900 μm, 930 μm, 950 μm and 980 μm.

Exemplarily, the distance h2 (i.e., the depth) from the opening K2 of the second accommodating cavity R2 to the bottom D2 of the second accommodating cavity R2 may be 220 μm, 230 μm, 240 μm, 250 μm, 260 μm, 270 μm, 280 μm and 290 μm.

Exemplarily, the diameter of the pattern enclosed by the outer contour of the bottom D1 of the first accommodating cavity R1 may be 3.1 mm, 3.2 mm, 3.3 mm, 3.5 mm, 3.8 mm, 4.0 mm, 4.2 mm, 4.5 mm, 4.8 mm and 4.9 mm.

Exemplarily, the diameter of the pattern enclosed by the outer contour of the bottom of the second accommodating cavity R2 may be 600 μm, 620 μm, 650 μm, 680 μm, 700 μm, 720 μm, 750 μm, 780 μm, 800 μm, 830 μm, 850 μm, 880 μm, 900 μm, 930 μm, 950 μm and 980 μm.

It should be noted that when the pattern enclosed by the outer contour of the bottom D1 of the first accommodating cavity R1 is an arc, a maximum diameter of the arc is 3 mm-5 mm, for example, 3.1 mm, 3.2 mm, 3.3 mm, 3.5 mm, 3.8 mm, 4.0 mm, 4.2 mm, 4.5 mm, 4.8 mm and 4.9 mm. When the pattern enclosed by the outer contour of the bottom of the second accommodating cavity R2 is an arc, a maximum diameter of the arc is 600 μm-1,000 μm, for example, 620 μm, 650 μm, 680 μm, 700 μm, 720 μm, 750 μm, 780 μm, 800 μm, 830 μm, 850 μm, 880 μm, 900 μm, 930 μm, 950 μm and 980 μm.

In at least one embodiment of the present application, an input voltage signal of the piezoelectric transducer 2 is 0.3 V-0.8 V, and the frequency of the input voltage signal is 150 kHz-400 kHz and 700 kHz-1,200 kHz.

It should be noted that the input voltage signals of the two piezoelectric transducers 2 are the same, and the frequencies and phases of the input voltage signals are also the same. The input voltage signal may be 0.4 V, 0.5 V, 0.6 V and 0.7 V. The input frequency may be 160 kHz, 180 kHz, 200 kHz, 220 kHz, 240 kHz, 260 kHz, 290 kHz, 300 kHz, 330 kHz, 350 kHz, 380 kHz, 720 kHz, 750 kHz, 780 kHz, 800 kHz, 820 kHz, 850 kHz, 880 kHz, 900 kHz, 960 kHz, 980 kHz, 1,000 kHz, 1,050 kHz, 1,080 kHz, 1,100 kHz and 1,150 kHz.

The input voltage signals of the two piezoelectric transducers 2 and the frequencies of the input voltage signals may be set according to the species of the cultured organoids and the different stages of the organoid development, and are not specifically limited herein.

In at least one embodiment of the present application, as shown in FIGS. 2 and 10, the culture structure further includes an enclosure structure 3. A part of the enclosure structure 3 is located between the main body structure 11 and the extending portion 12, and the enclosure structure 3 is disposed around the plurality of accommodating structures R. FIG. 10 is a schematic diagram of a cross-sectional structure along an M3M4 direction in FIG. 2.

In an exemplary embodiment, the enclosure structure 3 and the first surface B1 form a third accommodating cavity. The third accommodating cavity is located at a side of the first accommodating cavity R1 away from the second accommodating cavity R2 and the size of the third accommodating cavity is larger than a size of the first accommodating cavity R1. When the organoid is further developed and has a larger size, the organoid may be transferred from the first accommodating cavity R1 to the third accommodating cavity, thereby facilitating the further development and nutrient uptake of the organoid, and also facilitating the removal of the organoid at the end of the culture.

The patterns enclosed by the edge of the opening and the contour of the bottom of the third accommodating cavity described above are not limited herein and for example, may be any one of a circle, an oval, a quadrangle (including a rectangle or a square), and a pentagon.

In an exemplary embodiment, the enclosure structure 3 may be prepared by using the same material as the culture plate 1.

In at least one embodiment of the present application, as shown in FIG. 10, a thickness h3 of the culture plate 1 is 1.8 mm-3.5 mm, a size h4 of the enclosure structure 3 in a thickness direction of the culture plate 1 is 7 mm-12 mm, and a size h5 of the enclosure structure 3 in a direction parallel to the plane where the culture plate 1 is located is 1.5 mm-3.5 mm.

Exemplarily, the thickness h3 of the culture plate 1 may be 2 mm, 2.5 mm and 3 mm. The size h4 (i.e., the height of the enclosure structure 3) of the enclosure structure 3 in the thickness direction of the culture plate 1 may be 8 mm, 9 mm, 10 mm and 11 mm. The size h5 (i.e., the thickness of the enclosure structure 3) of the enclosure structure 3 in the direction parallel to the plane where the culture plate 1 is located may be 2 mm, 2.5 mm, 2.8 mm, 3.0 mm and 3.3 mm.

In an exemplary embodiment, a size of a planar pattern of the piezoelectric transducer 2 is less than the size of the planar pattern of the extending portion 2.

Exemplarily, the planar pattern of the main body structure 11 may be a square, a side length of the square may be 50 mm±0.5 mm, and the planar pattern of the extending portion 2 may be a rectangle, a long side of the rectangle may be 50 mm±0.5 mm.

The corresponding size and shape of the culture structure provided by the embodiment of the present application may be adjusted according to actual requirements, thereby various types of application scenarios are matched, for example, that the culture of different types of organoids is matched. The culture structure of the embodiment of the present application has a simple overall structure and a low preparation cost, and may be prepared in batches by using a processing process, which facilitates large-scale popularization and application.

A culture method is provided by embodiments of the present application, which is applied to the foregoing culture structure. The culture structure includes a culture plate 1 and a vibration signal generating component, and the vibration signal generating component is disposed on the culture plate 1. The culture plate 1 includes a plurality of accommodating structures R. The plurality of accommodating structures R are configured to accommodate culture solution, and the plurality of accommodating structures include a first accommodating cavity R1 and at least one second accommodating cavity R2 located at the bottom of the first accommodating cavity R1. An orthographic projection of the vibration signal generating component on the culture plate 1 does not overlap with the plurality of accommodating structures R. The method includes:
S801, generating the vibration signal,
wherein a voltage signal may be applied to the vibration signal generating component, and the vibration signal generating component generates the vibration signal according to the voltage signal; and
S802, driving, by the vibration signal, the culture solution in the plurality of accommodating structures to move.

According to the culture structure provided by the embodiments of the present application, the vibration signal generated by the vibration signal generating component drives the culture solution in the plurality of accommodating structures R to move. During the organoid growth process, sufficient oxygen may be provided to the organoids, and the oxygen and nutrients in the culture solution are balanced, so that the organoids cultured in the culture solution develop completely to solve the technical problem of organoid cell death caused by lack of oxygen or nutrients in the center during the organoid growth process.

In at least one embodiment of the present application, S802: driving, by the vibration signal, the culture solution in the plurality of accommodating structures R to move includes:
S8021, in a first stage, configuring the second accommodating cavity R2 to further accommodate a to-be-cultured object, a frequency of an input voltage signal of the vibration signal generating component being 150 kHz-400 kHz, and driving, by the vibration signal, the to-be-cultured object and the culture solution to move; and
S8022, in a second stage, configuring the first accommodating cavity R1 to accommodate the to-be-cultured object, the frequency of the input voltage signal of the vibration signal generating component being 700 kHz-1,200 kHz, and driving, by the vibration signal, the to-be-cultured object and the culture solution to move.

The development degree and size of the to-be-cultured object in the second stage are greater than the development degree and size of the to-be-cultured object in the first stage. The to-be-cultured object may include a cell and an organoid, for example, a brain organoid.

In the related art, the culture process of the organoid involves transfer through three culture plates. Firstly, small spheres are formed in a 96-well round-bottom culture plate, developed into embryoid bodies, and then transferred to a 24-well plate for further differentiation and development. After further growth, they are transferred to a 6-well plate for the final shaker oscillation development. That is, the traditional culture process of the organoid involves 3 transfers, which is very prone to organoid loss during the transfer processes.

In the embodiment of the present application, since the accommodating structure R includes a first accommodating cavity R1 and at least one second accommodating cavity R2 located at the bottom of the first accommodating cavity R1, the organoid is cultured and grown in the second accommodating cavity R2 during the first stage of the organoid growth process, and in the second stage, the organoid is cultured and grown in the first accommodating cavity R1, the loss and damage of the organoid caused by the transfer of the organoid at different development stages are avoided and the culture efficiency is improved.

A culture chip is provided by embodiments of the present application, which includes the foregoing culture structure.

In the culture chip provided in the embodiment of the present application, since the accommodating structure R includes a first accommodating cavity R1 and at least one second accommodating cavity R2 located at the bottom of the first accommodating cavity R1, it is possible to carry out the multi-stage culture of the organoid in the culture structure during the organoid growth process, the loss and damage of the organoid caused by the transfer of the organoid at different development stages are avoided and the culture efficiency is improved.

The above are merely particular embodiments of the present application, and the protection scope of the present application is not limited thereto. All of the variations or substitutions that a person skilled in the art can easily envisage within the technical scope disclosed by the present application should fall within the protection scope of the present application. Therefore, the protection scope of the present application should be subject to the protection scope of the claims.

1. A culture structure, comprising: a culture plate and a vibration signal generating component, wherein the vibration signal generating component is disposed on the culture plate;
the culture plate comprises a plurality of accommodating structures; the plurality of accommodating structures are configured to accommodate culture solution, and the plurality of accommodating structures comprise a first accommodating cavity and at least one second accommodating cavity located at a bottom of the first accommodating cavity; a volume of the first accommodating cavity is greater than a volume of the at least one second accommodating cavity; and
the vibration signal generating component is configured to generate a vibration signal; an orthographic projection of the vibration signal generating component on the culture plate does not overlap with the plurality of accommodating structures, and the vibration signal is for driving the culture solution in the plurality of accommodating structures to move.

2. The culture structure according to claim 1, wherein the culture plate comprises a main body structure and an extending portion located at at least one side of the main body structure; the plurality of accommodating structures are disposed on the main body structure, and the vibration signal generating component is disposed on the extending portion; and
the extending portion is connected to the main body structure, and the vibration signal acts on the main body structure through the extending portion.

3. The culture structure according to claim 2, wherein the vibration signal generating component comprises at least two piezoelectric transducers; the at least two piezoelectric transducers are disposed on the extending portion, and propagation directions of vibration signals generated by the at least two piezoelectric transducers intersect in a plane where the culture plate is located.

4. The culture structure according to claim 3, wherein the vibration signal generating component comprises two piezoelectric transducers, and in the plane where the culture plate is located, an included angle formed by the propagation directions of the vibration signals generated by the two piezoelectric transducers is greater than or equal to 80° and less than or equal to 100°.

5. The culture structure according to claim 4, wherein the culture plate comprises two extending portions, and the two extending portions are located at two adjacent sides of the main body structure, and the two extending portions are connected.

6. The culture structure according to claim 4, wherein the culture plate comprises a first surface, the first accommodating cavity comprise a bottom and an opening, and the second accommodating cavity comprise a bottom and an opening; a plane where the opening of the first accommodating cavity is located is coplanar with the first surface, and a plane where the opening of the second accommodating cavity is located is coplanar with the bottom of the first accommodating cavity;
wherein in a direction perpendicular to the first surface, a distance from the opening of the first accommodating cavity to the bottom of the first accommodating cavity is greater than a distance from the opening of the second accommodating cavity to the bottom of the second accommodating cavity.

7. The culture structure according to claim 6, wherein the second accommodating cavity comprises a second axis, and an included angle between the second axis of the second accommodating cavity and the first surface is greater than 0° and less than or equal to 90°.

8. The culture structure according to claim 7, wherein the included angle between the second axis of the second accommodating cavity and the first surface is substantially 90°, and an area of an orthographic projection of the bottom of the second accommodating cavity on the first surface is less than an area of a region enclosed by an orthographic projection of an edge of the opening of the second accommodating cavity on the first surface.

9. The culture structure according to claim 7, wherein the included angle between the second axis of the second accommodating cavity and the first surface is greater than 0° and less than 90°, and an area of an orthographic projection of the bottom of the second accommodating cavity on the first surface is less than or equal to an area of a region enclosed by an orthographic projection of an edge of the opening of the second accommodating cavity on the first surface.

10. The culture structure according to claim 8, wherein the first accommodating cavity comprises a first axis, and an included angle between the first axis of the first accommodating cavity and the first surface is substantially equal to the included angle between the second axis of the second accommodating cavity and the first surface; an area of a region enclosed by an orthographic projection of an outer contour of the bottom of the first accommodating cavity on the first surface is less than or equal to an area of a region enclosed by an orthographic projection of an edge of the opening of the first accommodating cavity on the first surface.

11. The culture structure according to claim 6, wherein an edge of the opening of the first accommodating cavity and an edge of the opening of the second accommodating cavity are arcs, an outer contour of the bottom of the first accommodating cavity and an outer contour of the bottom of the second accommodating cavity are arcs, and a side wall of the first accommodating cavity and a side wall of the second accommodating cavity are arc surfaces.

12. The culture structure according to claim 11, wherein the edge of the opening of the first accommodating cavity and the edge of the opening of the second accommodating cavity are circles, and the outer contour of the bottom of the first accommodating cavity and the outer contour of the bottom of the second accommodating cavity are circles;
each of the first accommodating cavity and the second accommodating cavity comprises two first regions; an intensity of the vibration signal received by the culture solution in each of the two first regions of the first accommodating cavity is greater than an intensity of the vibration signal received by the culture solution in other regions of the first accommodating cavity; an intensity of the vibration signal received by the culture solution in each of the two first regions of the second accommodating cavity is greater than an intensity of the vibration signal received by the culture solution in other regions of the second accommodating cavity; each circle comprises a first diameter and a second diameter, and the first diameter and the second diameter are orthogonal; an extending direction of the first diameter is consistent with the propagation direction of the vibration signal generated by one of the two piezoelectric transducers, and an extending direction of the second diameter is consistent with the propagation direction of the vibration signal generated by the other piezoelectric transducer; and
the two first regions are located at a same side of the first diameter, and orthographic projections of the two first regions on the first surface are symmetrically distributed about the second diameter.

13. The culture structure according to claim 12, wherein in the direction perpendicular to the first surface, the distance from the opening of the first accommodating cavity to the bottom of the first accommodating cavity ranges from 250 μm to 1,000 μm, and the distance from the opening of the second accommodating cavity to the bottom of the second accommodating cavity ranges from 200 μm to 300 μm; and
a diameter of a pattern enclosed by the outer contour of the bottom of the first accommodating cavity ranges from 3 mm to 5 mm, and a diameter of a pattern enclosed by the outer contour of the bottom of the second accommodating cavity ranges from 600 μm to 1,000 μm.

14. The culture structure according to claim 2, wherein the culture structure further comprises an enclosure structure; a part of the enclosure structure is located between the main body structure and the extending portion, and the enclosure structure is disposed around the plurality of accommodating structures.

15. The culture structure according to claim 14, wherein a thickness of the culture plate is 1.8 mm-3.5 mm, a size of the enclosure structure in a thickness direction of the culture plate is 7 mm-12 mm, and a size of the enclosure structure in a direction parallel to the plane where the culture plate is located is 1.5 mm-3.5 mm.

16. A culture method, applied to the culture structure according to claim 1, wherein the culture structure comprises the culture plate and the vibration signal generating component, and the vibration signal generating component is disposed on the culture plate; the culture plate comprises the plurality of accommodating structures; the plurality of accommodating structures are configured to accommodate the culture solution and the plurality of accommodating structures comprise the first accommodating cavity and the at least one second accommodating cavity located at the bottom of the first accommodating cavity; the orthographic projection of the vibration signal generating component on the culture plate does not overlap with the plurality of accommodating structures; the method comprises:
generating the vibration signal; and
driving, by the vibration signal, the culture solution in the plurality of accommodating structures to move.

17. The culture method according to claim 16, wherein the step of driving, by the vibration signal, the culture solution in the plurality of accommodating structures to move comprises:
in a first stage, configuring the second accommodating cavity to further accommodate a to-be-cultured object, a frequency of an input voltage signal of the vibration signal generating component being 150 kHz-400 kHz, and driving, by the vibration signal, the to-be-cultured object and the culture solution to move; and
in a second stage, configuring the first accommodating cavity to accommodate the to-be-cultured object, the frequency of the input voltage signal of the vibration signal generating component being 700 kHz-1,200 kHz, and driving, by the vibration signal, the to-be-cultured object and the culture solution to move.

18. A culture chip, comprising the culture structure according to claim 1.

19. The culture structure according to claim 9, wherein the first accommodating cavity comprises a first axis, and an included angle between the first axis of the first accommodating cavity and the first surface is substantially equal to the included angle between the second axis of the second accommodating cavity and the first surface; an area of a region enclosed by an orthographic projection of an outer contour of the bottom of the first accommodating cavity on the first surface is less than or equal to an area of a region enclosed by an orthographic projection of an edge of the opening of the first accommodating cavity on the first surface.

20. The culture structure according to claim 7, wherein an edge of the opening of the first accommodating cavity and an edge of the opening of the second accommodating cavity are arcs, an outer contour of the bottom of the first accommodating cavity and an outer contour of the bottom of the second accommodating cavity are arcs, and a side wall of the first accommodating cavity and a side wall of the second accommodating cavity are arc surfaces.
